(19) United States
(12) Patent Application Publication
(10) Pub. No.: US 2022/0347393 A1
(43) Pub. Date: Nov. 3, 2022

(54) ADMINISTRATION MECHANISM FOR A MEDICAMENT DELIVERY DEVICE

(71) Applicant: SHL MEDICAL AG, ZUG (CH)

(72) Inventor: Hsueh-Yi Chen, New Taipei City (TW)

(21) Appl. No.: 17/634,640

(22) PCT Filed: Aug. 19, 2020
(86) PCT No.: PCT/EP2020/073209

§ 371 Date: Feb. 11, 2022

(30) Foreign Application Priority Data
Sep. 2, 2019 (EP) 19194908.0

(51) Int. Cl. A61M 5/315; A61M 5/20; A61M 5/32

(52) U.S. Cl. CPC A61M 5/31586; A61M 5/2033; A61M 5/20; A61M 5/3204; A61M 5/31505; A61M 2005/2013; A61M 2005/208; A61M 2005/2086; A61M 2005/3152

(57) ABSTRACT

A medicament delivery device is presented having a proximal disposable part arranged with a body and a delivery member cover telescopically arranged in relation to the body wherein the proximal disposable part is configured to receive a medicament container. A distal reusable power pack is detachably arranged with the proximal disposable part such that the delivery member cover has an extension position in which the delivery member cover is extended in the proximal direction to cover a medicament delivery member of the medicament container, and a retraction position in which the delivery member cover is retracted in the distal direction to expose said medicament delivery member. The distal reusable power pack can have a power unit, a rotational drive unit and a plunger unit, where the rotational drive unit is powered by the power unit and is configured to move the plunger unit upon being triggered.

### CROSS REFERENCE TO RELATED APPLICATIONS

The present application is a U.S. National Phase Application pursuant to 35 U.S.C. § 371 of International Application No. PCT/EP2020/073209 filed Aug. 19, 2020, which claims priority to European Patent Application No. 19194908.0 filed Sep. 2, 2019. The entire disclosure contents of these applications are herewith incorporated by reference into the present application.

### TECHNICAL FIELD

The present disclosure generally relates to medical devices. In particular, it relates to an administration mechanism for a medicament delivery device, and to a medicament delivery device comprising an administration mechanism.

### BACKGROUND

Medicament delivery devices such as auto-injectors are generally known for the self-administration of a medicament by patients without formal medical training. As just one example, those patients suffering from diabetes may require repeated injections of insulin. Other patients may require regular injections of other types of medicaments, such as a growth hormone. Reusable auto-injectors are commonly used by patients who require repeated injections.

Reusable auto-injectors often have two parts which are interconnected during use. One disposable part often receives a medicament container which contains a medicament, such as a cartridge or medicament container. The other reusable part often comprises a power unit such as a spring or a motor and thus can be re-used during a number of injections.

WO 2012/085030 A1 discloses a reusable auto-injector with a front-end device receives a medicament container and a reusable back-end device connected to the front-end device. The reusable back-end device comprises an electric motor configured to be activated to translate the medicament container within the front-end device parallel to an axis of the substantially cylindrical front-end device and to expel the medicament contained in the medicament container. A plunger is configured to connect to a stopper of the medicament container and upon being activated, moves forward to expel the medicament contained in the medicament container through a needle of the medicament container. A plurality of control elements are used to activate and control a variety of features of the auto-injector, such as activating and de-activating the electric motor that axially translates the plunger to insert and/or retract the needle and to inject the dose of the medicament.

WO 2016/091840 A1 discloses an auto-injector with a medicament cartridge received in a housing. The body comprises a main spring, a dial gear, a plunger for abutting a bung of the medicament cartridge. The main spring is configured to act directly on the proximal surface of the plunger. It is the main spring acting on the plunger that drives the bung axially in order to deliver medicament.

Configurations disclosed in WO 2012/085030 A1 and WO 2016/091840 A1 expose the needle before the patient triggers the power unit, which raises a risk of injuring the patient. It requires the patient to find a proper injection angle the injection depth on the injection site during injection. Further, since configurations disclosed in WO 2012/085030 A1 and WO 2016/091840 A1 are both used a button positioned on the outer surface of the body of the auto-injector, there is a risk of premature/unintentional trigger of the auto-injector by accidentally grapping or touching on the trigger button.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

Further, the term “longitudinal”, refers to a direction extending from the proximal end to the distal end and through the device or components thereof in the direction of the longest extension of the device and/or component.

Similarly, the terms “orthogonal” refers to a direction generally perpendicular to the longitudinal direction.

An object of this disclosure is to provide a simple and reliable way of activation of a reusable medicament delivery device, by introducing a delivery member cover which is arranged on the disposable part, and configured to trigger the power unit on the reusable part while moving in the distal direction e.g. being pressed against to the injection site.

According to an aspect of the present disclosure and concept, the object is achieved by a medicament delivery device according to claim 1.

There is hence provided a medicament delivery device comprising: a proximal disposable part arranged with a body and a delivery member cover telescopically arranged in relation to the body wherein the proximal disposable part is configured to receive a medicament container; a distal reusable power pack detachably arranged with the proximal disposable part; wherein said delivery member cover has an extension position in which the delivery member cover is extended in the proximal direction to cover a medicament delivery member of the medicament container, and a retraction position in which the delivery member cover is retracted in the distal direction to expose said medicament delivery member; wherein said distal reusable power pack comprises a power unit, a rotational drive unit and a plunger unit; wherein said rotational drive unit is powered by said power unit and is configured to move said plunger unit upon being triggered; wherein said plunger unit is configured to move a stopper of the medicament container in the proximal direction upon being moved by said rotational drive unit, so that a medicament contained in the medicament container is ejected; wherein said delivery member cover comprises a trigger configured to trigger said rotational drive unit to move said plunger unit when said delivery member cover is moved distally to the retraction position.

According to one embodiment the reusable medicament delivery device is a variable dose device or a fixed dose device.

According to one embodiment the reusable medicament delivery device can be an injection device, a needleless jet injection device, an inhalation device or a nasal sprayer.

According to one embodiment the distal reusable power pack is detachably arranged with the proximal disposable part through a connection, such as a snap fitting, thread engagement, bayonet engagement, ratchet teeth connection or magnetic connection.

According to one embodiment the distal reusable power pack is configured to be used with one of the plurality of interchangeable proximal disposable parts.

According to one embodiment the distal reusable power pack and the proximal disposable part further comprises a key element and a counter key element; therefore, only the proper proximal disposable part is able to be connected to the distal reusable power pack.

According to one embodiment the distal reusable power pack comprises a printed circuit board (PCB), arranged with a processor, a switch and a signal unit.

According to one embodiment the distal reusable power pack further comprises a data receiver configured to read the data from a tag on the proximal disposable part.

According to one embodiment the data receiver can be a contactless data receiver, such as RFID reader, NFC antenna or barcode reader.

According to one embodiment the signal unit can be a sound indicator, a vibration indicator or a light indicator; and is configured to indicate a user with a signal regarding to the status of the medicament delivery device operation.

According to one embodiment the proximal disposable part configured to receive a medicament container containing a medicament.

According to one embodiment the medicament delivery member is a spray nozzle.

According to one embodiment the medicament delivery member is an injection needle.

According to one embodiment the injection needle is integral with the medicament container.

According to one embodiment the medicament container is a cartridge, and arranged with a double end injection needle; which is not in a fluid communication with the cartridge before the user intend to perform the injection.

According to one embodiment the double end injection needle is configured to be in a fluid communication with the cartridge by the removal of a protective cap; the protective cap is configured to fully cover the injection needle before its removal.

According to one embodiment the proximal disposable part further comprises a medicament container housing to contain the medicament container.

According to one embodiment said power unit is a spring, the spring being configured to deliver a spring force along its circumferential direction.

According to one embodiment the spring can be a torsion spring, a spiral spring or a clock spring.

According to one embodiment said power unit is a motor.

According to one embodiment said rotational drive unit comprises a rotational gear wheel configured to rotate related to an orthogonal axis of relation to the distal reusable power pack.

According to one embodiment said rotational drive unit comprises a drive nut configured to rotate related to a longitudinal axis of the distal reusable power pack.

According to one embodiment said rotational drive unit comprises an insert rotationally locked to the distal reusable power pack.

According to one embodiment said plunger has a plurality of teeth, said plunger unit being arranged to connect with said gear wheel by means of said teeth.

According to one embodiment said plunger unit is configured with plurality segments.

According to one embodiment said plurality segments of plunger unit are connected to each other through hinge connection.

According to one embodiment said plunger unit is a wire.

According to one embodiment said plunger unit comprises a thread on its external surface.

According to one embodiment said plunger unit is configured with a slot on its external surface and configured to engage with either the drive nut or the insert.

According to one embodiment said distal reusable power pack comprises a housing, said housing being configured to receive said rotational drive unit and said power unit, wherein said housing further comprises a locking element configured to lock said rotational drive unit from rotating in relation to said housing in a locking position.

According to one embodiment wherein said trigger is configured to release said locking element to move out from the locking position, thereby triggering said rotational drive unit to move the plunger unit.

According to one embodiment said distal reusable power pack further comprises a reset knob arranged to rotate the rotational drive unit against the restoring force of the spring.

According to one embodiment the rotation of said reset knob actuates said plunger unit to move in the distal direction in relation to said proximal disposable part by the connection between the plunger unit and the rotational drive unit.

According to one embodiment the rotation of said reset knob is formed a part of the distal reusable power pack.

According to one embodiment the rotation of said reset knob is an independent element releasably attached to the distal reusable power pack.

According to one embodiment said motor is configured to deliver a rotation to the rotational drive unit.

According to one embodiment the rotation that is delivered by said motor is controlled by the switch.

According to one embodiment the distal reusable power pack further comprises a switch; wherein said trigger is configured to trigger said motor by switching the switch.

According to one embodiment said motor is configured to reciprocate the movement of said plunger unit.

According to one embodiment the reciprocating movement of said motor is controlled by the switch so as the reciprocating movement of said plunger unit.

According to one embodiment said delivery member cover comprises a rib and said proximal disposable part comprises a rotator configured with a locking tongue, said rotator being configured to be rotated by a linear distal movement of the delivery member cover, wherein said rotation aligns the locking tongue with said rib, so that the delivery member cover is prevented from moving to the retraction position.

According to one embodiment said rotator being configured to be rotated by a linear proximal movement of the delivery member cover, wherein said rotation aligns the locking tongue with said rib, so that the delivery member cover is prevented from moving to the retraction position.

According to one embodiment the rib is positioned in the guide track, and moves along the guide track.

According to one embodiment the guide track is arranged on the outer surface of the rotator.

According to one embodiment the rib is arranged on the inner surface of the delivery member cover.

According to one embodiment the guide track is arranged on the inner surface of the rotator.

According to one embodiment the rib is arranged on the outer surface of the delivery member cover.

According to one embodiment the rib can also act as the trigger.

According to one embodiment the rib can also act as the abutment.

Other aspects, features, and advantages will be apparent from the summary above, as well as from the description that follows, including the figures and the claims.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the present concept will now be described, by way of example, with reference to the accompanying drawings, in which:

FIG. 1A displays a first embodiment of a medicament delivery device;

FIG. 1B displays an example of a medicament delivery device with a proximal disposable part and a distal reusable power pack;

FIG. 2 displays an exploded view of the distal reusable power pack in FIG. 1b;

FIG. 3 displays detail views of components in the embodiment of FIG. 1b;

FIG. 4 displays a perspective view with the locking element;

FIG. 5 displays an another perspective view of the distal reusable power pack connects to the proximal disposable part;

FIG. 6 displays detail views of components in the proximal disposable part;

FIG. 7 displays a second embodiment of a medicament delivery device;

FIG. 8 displays an example of a medicament delivery device with a proximal disposable part and a distal reusable power pack;

FIG. 9 displays an exploded view of the distal reusable power pack in FIG. 8;

FIG. 10 displays a perspective view of the distal reusable power pack connects to the proximal disposable part;

FIG. 11 displays a third embodiment of a medicament delivery device;

FIG. 12 displays a perspective view of the distal reusable power pack; and

FIG. 13 displays a perspective view of the distal reusable power pack connects to the proximal disposable part.

### DETAILED DESCRIPTION

The present concept will now be described more fully hereinafter with reference to the accompanying drawings, in which exemplifying embodiments are shown. The present concept may, however, be embodied in many different forms and should not be construed as limited to the embodiments set forth herein; rather, these embodiments are provided by way of example so that this disclosure will be thorough and complete, and will fully convey the scope of the present concept to those skilled in the art. Like numbers refer to like elements throughout the description.

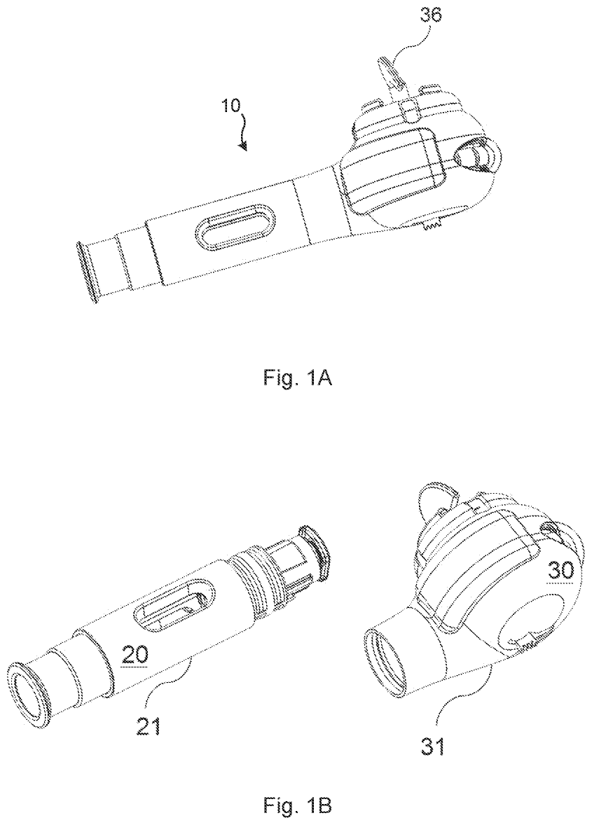

FIG. 1 generally illustrates a first embodiment of the medicament delivery device 10 for performing injections. The medicament delivery device 10 comprises a proximal end, distal end and a longitudinal axis extending between the proximal end and the distal end. The medicament delivery device 10 has a proximal disposable part 20 and a distal reusable power pack 30. The distal reusable power pack 30 is detachably arranged with the proximal disposable part 20.

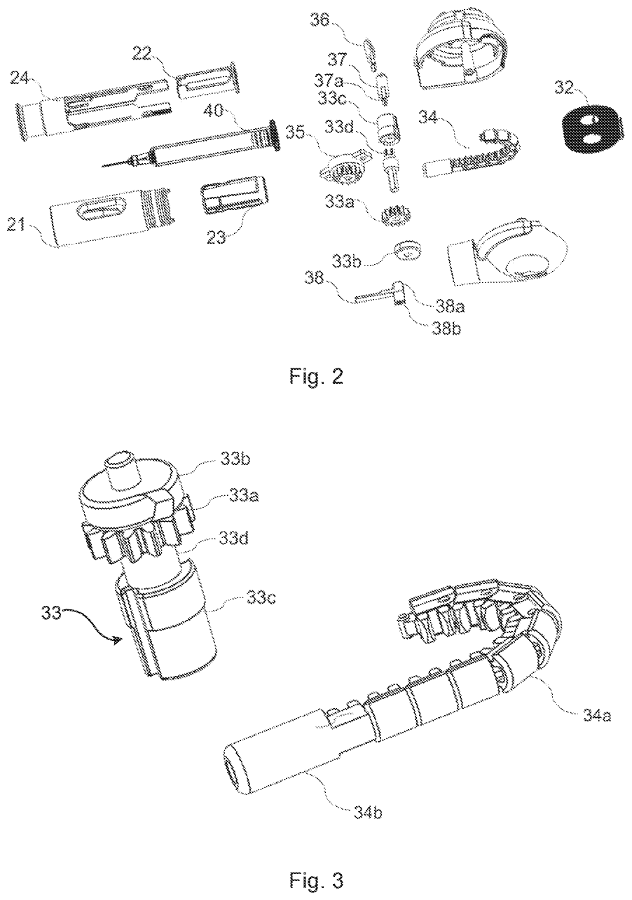

FIG. 2 and FIG. 3 illustrates detail components of the proximal disposable part 20 and the distal reusable power pack 30. The proximal disposable part 20 comprises a body 21, a medicament container housing, 22 a rotator 23 and a delivery member cover 24, which is telescopic in relation to the body 21 and movable along a longitudinal axis. The medicament container housing 22 is configured to receive a medicament container 40 therein. The medicament container 40 is configured to container a medicament and arranged with a medicament delivery member arranged, the medicament delivery member is arranged on the proximal end of the proximal disposable part 20, and arranged on the proximal end of the medicament delivery device 10 when the proximal disposable part is attached with the distal reusable part pack 30. The proximal disposable part 20 further comprises a protective cap (not show) to cover the medicament delivery member of the medicament container 40, and a delivery member cover biasing member (not show), configured to bias the delivery member cover 24 in the proximal direction into a delivery member cover 24 extension position. So that the medicament delivery member of the medicament container 40 is covered before and after an operation of the medicament delivery sequence, such like before and after the medicament delivery device 10 has been placed on/removed from an injection site. The biasing force can be overcome by pressing the delivery member cover in the distal direction, so that in a delivery member cover retraction position, the medicament delivery member of the medicament container 40 is exposed. The biasing member could be a spring or a flexible arm that is arranged between the body 21 and the delivery member cover 24.

The distal reusable power pack 30 comprises a housing 31, a power unit 32, a rotational drive unit 33, a plunger unit 34 and a damper gear 35.

The plunger unit 34 is configured to actuate a stopper of the medicament container 40 during the medicament delivery operation, so that the stopper of the medicament container 40 is moved forward along the longitudinal axis of the medicament delivery device 10, and a medicament contained in the medicament container 40 is expelled. The rotational drive unit 33 is configured to contact the plunger unit 34 and moves the plunger unit 34 in a longitudinal direction (FIG. 5). The power unit 32 is an element accumulated with energy and configured to transit the accumulated energy to the rotational drive unit (33).

FIG. 3 illustrates a detail of the rotational drive unit 33 and the plunger unit 34. The rotational drive unit 33 comprises a rotational gear wheel 33a which is rotationally fixed to a rotational cam ring 33b. The plunger unit 34 comprises a plunger body 34a and a plunger head 34b. The plunger body 34a is axially fixed to a plunger head 34b. The plunger body 34a comprises plurality teeth, which are configured to interact with counter teeth of the rotational gear wheel 33a. The counter teeth are configured to mesh or engage with the teeth. The plunger head 34b is configured to confront or meet with and move a rubber stopper of the medicament container 40 for expelling a dose of medicament contained in the medicament container 40. The rotational drive unit 33 further comprises a spring shaft 33c and a central shaft 33d.

The power unit 32 is in the present example a power spring is fixed to the housing 31 at one end and fixed to the spring shaft 33c at the other end. The spring shaft 33c receives and is rotationally fixed to the central shaft 33d. The spring shaft 33c and the central shaft 33d are both rotatable and will be rotated together in relation to the housing. In an alternative embodiment, the central shaft 33d and the spring shaft 33c can be integral together as one single component.

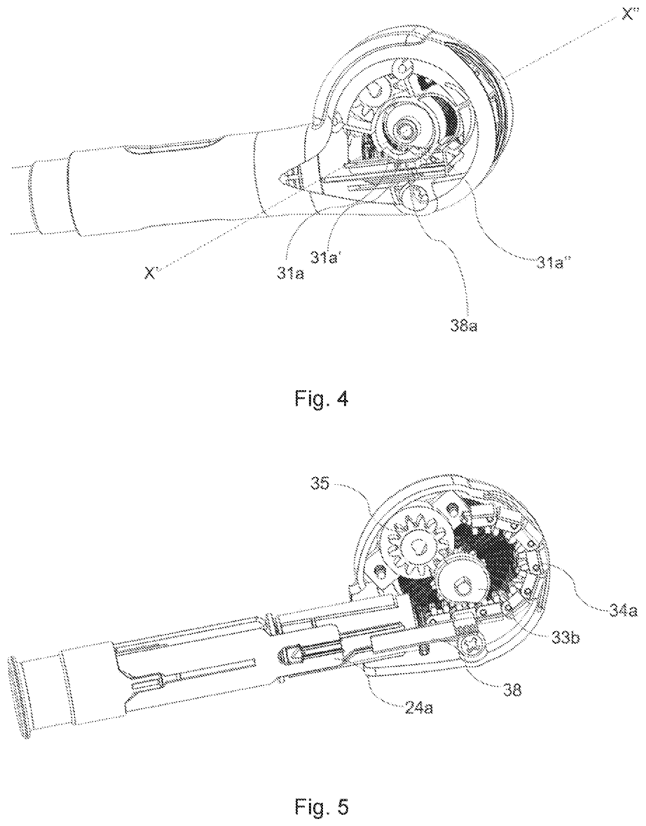

The central shaft 33d is a central shaft of the rotational gear wheel 33a and the rotational cam ring 33b. The central shaft 33d is rotationally fixed to both the rotational gear wheel 33a and the rotational cam ring 33b; so that the rotational gear wheel 33a and the rotational cam ring 33b is configured to rotate together with the central shaft 33d and the spring shaft 33c. Since the rotational gear wheel 33a is rotationally fixed to the central shaft 33d, so once the power spring 32 is released, the spring force will rotate the spring shaft 33c, the central shaft 33d and the rotational gear wheel 32 all together; therefore, the rotational gear wheel 33a is configured to rotate about an axis X′-X″ of the distal reusable power pack 30 shown in FIG. 4 under the force of the power spring 32.

FIG. 5 shows the damper gear 35 connected to the rotational gear wheel 33a by teeth on both the damper gear 35 and the rotational gear wheel 33a. The damper gear 35 is rotationally arranged in relation to the rotational gear wheel 33a, and able to rotate about an axis offset to the axis X′-X″ of the rotational gear wheel 33a.

The distal reusable power pack 30 also comprises a reset knob 36 fixedly connected to a reset shaft 37, shown in FIG. 2. The reset shaft 37 comprises a key 37a configured to be received by at least one of a first receiving portion of the spring shaft 33c or a second receiving portion of the central shaft 33d; or the key 37a may be received by both the first receiving portion and the second receiving portion. In a preferred embodiment, the key 37a comprises a radial outwardly extending protrusion and the spring shaft 33c comprises a first groove; the central shaft 33d comprises a second groove; the radial outwardly extending protrusion of the key 37a is received by both the first groove and the second groove. So that the reset shaft 37 can be rotated together with the spring shaft 33c and the central shaft 33d by a torque applied on the reset knob 36. Since the central shaft 33d and the spring shaft 33c is rotationally fixed together, the radial outwardly extending protrusion of the key 37a can be received by only one of the first or second groove; and the reset shaft 37 can still be rotated together with the spring shaft 33c and the central shaft 33d by a torque applied on the reset knob 36. In a preferred embodiment, the reset knob 36 and the reset shaft 37 are formed as a part of the distal reusable power pack 30, such that a user of the distal reusable power pack 30 can easily reset the power pack. In an alternative embodiment, the reset knob 36 and the reset shaft 37 are an independent resetting assembly in relation to the distal reusable power pack 30 and can be removable attached to the distal reusable power pack 30; so that the distal reusable power pack 30 may be designed as only specific group of people, e.g. doctor or nurse; can reset it. The reset knob 36 is positioned outside the housing 31 and is accessible by a user for interaction, see FIG. 1a.

The distal reusable power pack 30 further comprises a sliding pin 38 and a locking element 31a. The sliding pin 38 comprises a blocking member 38a and a sliding protrusion 38b. As illustrated in FIG. 4, the locking element 31a is fixedly arranged in relation to the housing 31 on one end, which is a fixed end, and freely arranged in relation to the housing on the other end, which is a free end. The locking element 31a comprises a flexible arm 31a′, extending between the fixed end and the free end, and a locking head 31a″ located on the free end. The locking head is configured to engage with a unidirectional cam on the edge of the rotational cam ring 33b when the flexible arm 31a′ is tensed by the blocking member 38a; and is configured to disengage with the unidirectional cam on the edge of the rotational cam ring 33b when the blocking member 38a is not interacting with the flexible arm 31a′, so the flexible arm is relaxed.

The proximal disposable part 20 is connected to the distal reusable power pack 30 through a connection between the body 21 and the housing 31.

FIG. 4 illustrates the medicament delivery device 10 in a locking position. In the locking position, the flexible arm 31a′ is bent to its tensional configuration and moves the locking head 31a″ close to the rotational cam ring 33b by the blocking member 38a of the sliding pin 38, so that the locking head 31a″ abuts the unidirectional cam on the edge of the rotational cam ring 33b. Since the rotational cam ring 33b is rotationally fixed to the rotational gear wheel 33a, rotation of the rotational gear wheel 33a under the force of the power spring 32 is blocked.

Once the user is to use the medicament delivery device 10, e.g. perform the injection, he/she will firstly connect and secure the proximal disposable part 20 to the distal reusable power pack 30 and remove the protective cap (not show). The user then presses the delivery member cover 24 against the injection site. The distal end of the delivery member cover 24 comprises a distal extending structure acts as a trigger 24a, distally moving the sliding pin 38 together as the delivery member cover 24 is moved distally (FIG. 5).

The distal movement of the sliding pin 38 causes misalignment between the blocking member 38a and the locking head 31a″, so that the flexible arm 31a′ is released to its relaxed state. The locking head 31a″ is thereby moved out of engaged with the unidirectional cam on the edge of the rotational cam ring 33b under the resilient force of the flexible arm 31a′.

Due to the disengagement between the locking element 31a and the rotational cam ring 33b, the rotational cam ring 33b is now free to rotate in relation to the housing 31. Because of the rotatable arrangement between the spring shaft 33c and the central shaft 33d in relation to the housing 31, and the rotationally fixed connection between the spring shaft 33c, the central shaft 33d, the rotational gear wheel 33a and the rotational cam ring 33b, once the rotational cam ring 33b is free to rotate in relation to the housing 31, the spring shaft 33c, the central shaft 33d, the rotational gear wheel 33a and the rotational cam ring 33b are rotated under the force of the power spring 32 along a first rotation direction.

The rotation of the rotational gear wheel 33a moves the plunger body 34a along the longitudinal axis in the proximal direction due to cooperation between the teeth and the counter teeth of the rotational gear wheel 33a and the plunger body 34a. The force of the power unit 32 is thereby able to be transmitted to and move the plunger unit 34 in the proximal direction. The plunger head 34b is able to move the stopper of the medicament container 40 forward, to eject the medicament contained in the medicament container 40.

After a predetermined dose of the medicament contained in the medicament container 40 has been ejected, the user removes the medicament delivery device 10 from the injection site, so that the delivery member cover 24 is moved proximally under the biasing force of the biasing member (not show) and covers the needle of the medicament container 40.

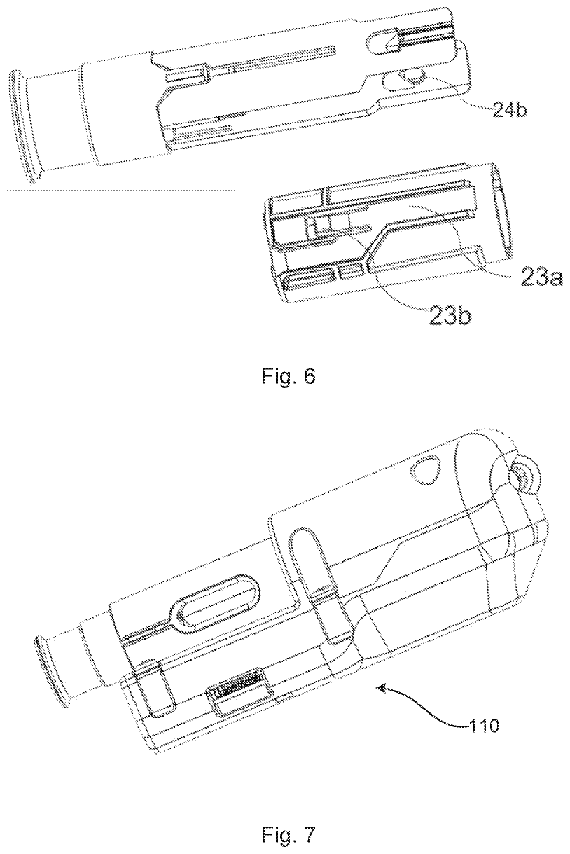

FIG. 6 shows the delivery member cover 24 and the rotator 23. The rotator 23 comprises a guide track 23a, comprises an initial proximal portion, an intermediate distal portion and a final proximal portion. The guide track further comprises a ramp, which is positioned between the initial proximal portion and the intermediate distal portion. The delivery member cover 24 comprises a rib 24b arranged on the distal part of the delivery member cover 24. The rib 24b is positioned on the initial proximal part of the guide track 23a at the beginning, which is before an operation of the medicament delivery has been initiated; the rib 24b is configured to be moved to the intermediate distal portion with the distal linear movement of the delivery member cover 24. While the rib 24b is moving to the intermediate distal portion from the initial proximal portion, the rib 24b interacts with the ramp. Since the delivery member cover 24 is rotationally locked in relation to the body 21, the interaction between the rib 24b and the ramp causes the rotator 23 to rotate. After the rotator has been rotated by the interaction between the rib 24b and the ramp, the rib 24b reaches to the intermediate distal portion of the track 23a. Upon the delivery member cover 24 moves to the proximal direction again, such like the force applied on the proximal end of the delivery member cover 24 has been remove, so that the delivery member cover is biased to the proximal direction. The proximal linear movement of the delivery member cover 24 moves the rib 24b from the intermediate distal portion to the final proximal portion of the guide track 23a. The rotator further comprises a locking tongue 23b, arranged between the intermediate distal portion and the final proximal portion of the guide track 23. So during the rib 24b moves from the intermediate distal portion to the final proximal portion of the guide track 23 the ribs 24b will move pass the locking tongue 23b, which the rib 24b will radially compress the locking tongue 23b and release the locking tongue 23b after the rib 24b reaching the final proximal portion of the guide track 23a. Once the rib 24b arrived the final proximal portion of the guide track 23a, the distal movement of the rib 24b is blocked by a proximal surface of the locking tongue 23b. Such that the further distal movement of the delivery member cover 24 is therefore prevented and the delivery member cover 24 is therefore cannot move to the retraction position. When the rib 24b is arrived the final proximal portion, the medicament delivery member 24 is moved into its extension position, such that the proximal end of the delivery member cover 24 is configured to completely surround the medicament delivery member of the medicament container 40; so that the exposure of the medicament delivery member is prevented.

The user now can remove the proximal disposable part 20 from the distal power unit 30, and reset the distal power unit 30 by firstly the user twisting the reset knob 36. The torque of the reset knob 36 causes the rotation of the reset shaft 37 along a second rotation direction, which is opposite with the first rotation direction. For example, the first rotation direction may be a clockwise direction, and the second rotation direction may then be an anti-clockwise direction. The reset shaft 37 is rotated with the spring shaft 33c and the central shaft 33d against the restoring force of the power spring 32, so that the power spring is tensioned again. The sliding pin 38 is slid back to the locking position by sliding a protrusion 38b of the sliding pin 38, so that the blocking member 38a, is aligned with the locking head 31a″ and bends the flexible arm 31a′ again. The sequence can alternatively be replaced by first sliding the sliding pin 38 back to the locking position, then twisting the reset knob 36.

Also, the rotational gear wheel 33a and the rotational cam ring 33b are rotated in the second rotation direction together with the spring shaft 33c and the central shaft 33d. The rotation of the rotational gear wheel 33a along the second rotation direction causes the distal movement of the plunger body 34a in relation to the proximal disposable part. The user may stop twisting when the spring has been fully tensed and the plunger body has been fully backed to its initial position.

The unidirectional cam on the edge of the rotational cam ring 33b is configured to engage with the locking head 31a″ in the first rotation direction, but is free to move relative to the locking head 31a″ in the second rotation direction.

Once the distal reusable power pack 30 has been fully reset and the user releases the reset knob 36, the spring shaft 33c, the central shaft 33d, the rotational gear wheel 33a and the rotational cam ring 33b will be rotated under the spring force until the unidirectional cam on the edge of the rotational cam ring 33b meets the locking head 31a″. The distal reusable power pack 30 is then in the locking position again.

The embodiment as described above is the activation mechanism for a reusable automatic medicament delivery device. However, the activation mechanism can also be used in a disposable automatic medicament delivery device. In that case, the reset knob 36 and the reset shaft 37 don't need to be included in the device arrangement.

Also, the activation mechanism can be used in an automatic medicament delivery device with a variable dose. In that case, the rotational cam ring 33b can be modified with variable unidirectional cams with different arc corresponding to variable dose selections, and the reset knob 36 can act as the dose setting knob.

The embodiment as described above discloses components for easy manufacturing. However, it can also be modified for easily assembling. For example, the spring shaft 33c and the central shaft 33d can be integral as one component, the rotational gear wheel 33a and the rotational cam ring 33b can be also integral as one component, or the spring shaft 33c, the central shaft 33d, the rotational gear wheel 33a and the rotational cam ring 33b can be integral as one component. According to one variation, the reset knob 36 and the reset shaft 37 can be integral as one component as well. Also, the trigger 24a could be an integral part of the delivery member cover 24 or a separate component attached to the delivery member cover 24, and the structure of the trigger 24a could be a rib, protrusion, recess or any other structure which is able to push the sliding pin 38 to move distally.

Also, the protrusion 38b described above can be arranged smaller/less significant for preventing the user from mistaking it for as a triggering button. The protrusion 38b could also be fully positioned in a recess of the housing 30, so that only the caregiver can reset the device or set a dose by a tool e.g. a key, for the user to inject. On the other hand, the protrusion 38b can be removed from the design, by replacing it with a spring or a biasing arm between the sliding pin 38 and the housing 31. The spring or biasing arm can be either integral as a part of housing 31 or a part of sliding pin 38 or a separate element, so that the sliding pin 38 will be biased back to the locking position and bend the flexible arm 31a′ again.

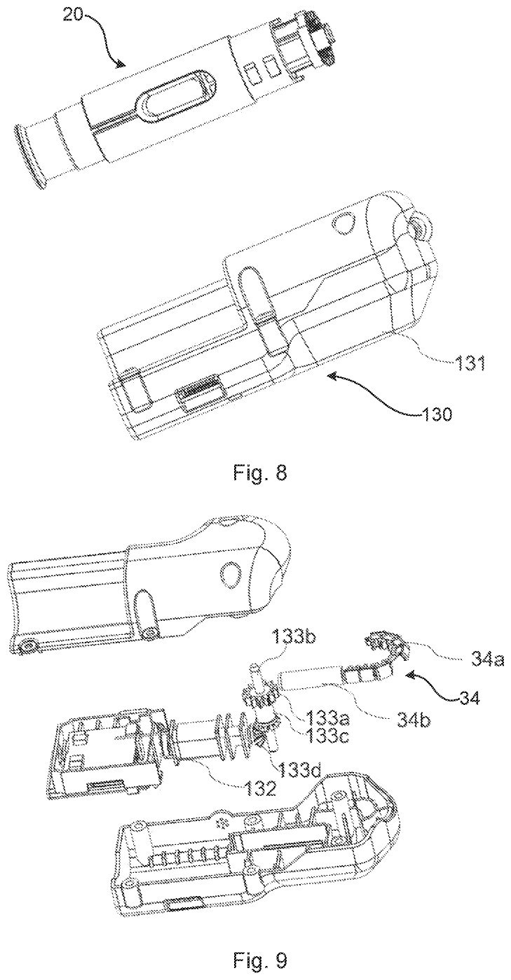

FIG. 7 and FIG. 8 illustrate a second embodiment of a medicament delivery device. The medicament delivery device 110 has a proximal disposable part 20 and a distal reusable power pack 130. The proximal disposable part 20 used in the second embodiment is the same as the proximal disposable part 20 used in the first embodiment, e.g. components, arrangements of components and functions are the same as described in the first embodiment. The proximal disposable part 20 can be connected to the distal reusable power pack 130 by the same connection as used in the first embodiment.

The distal reusable power pack 130 shown in FIG. 9, comprises a housing 131, a power unit 132, a rotational drive unit 133, and a plunger unit 34. The plunger unit 34 used in the second embodiment is the same as the plunger unit 34 used in the first embodiment.

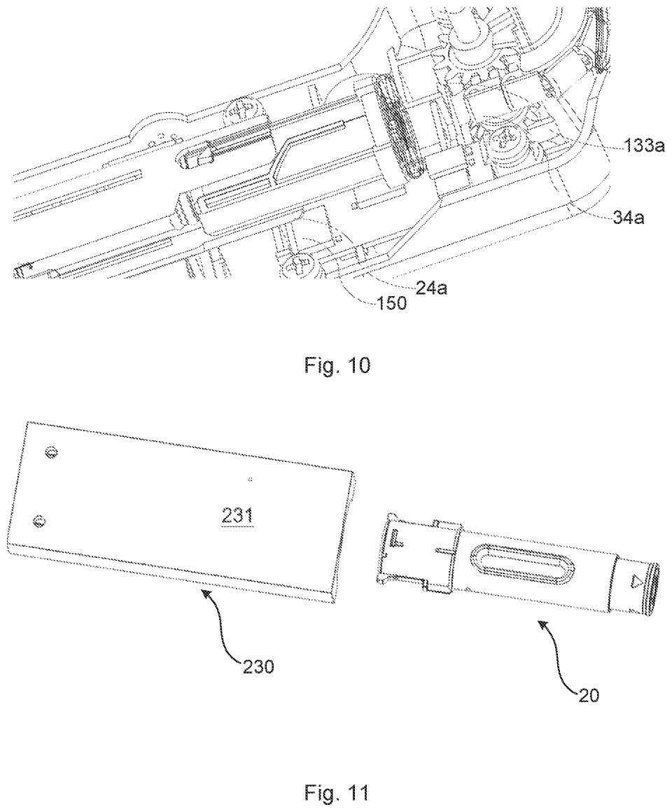

FIG. 10 shows the rotational drive unit 133 configured to connect the plunger unit 34 and configured to move the plunger unit 34 along the longitudinal axis. The power unit 132 is an element accumulated with the energy that is to be transmitted to the rotational drive unit 133.

FIG. 9 shows the rotational drive unit 133 comprises a gear wheel 133a rotationally locked to a central shaft 133b in the central part. The central shaft 133b is rotationally locked with a second transmission gear 133c. The second transmission gear 133c, the central shaft 133b and the rotational drive unit 133 are able to rotate together. The rotational drive unit 133 further comprises a first transmission gear 133d.

The power unit 132 is a motor, configured to deliver a torque trough an output end, so that once the motor is switched on, the output end will start to rotate. The output end of the motor is a first transmission gear 133d, configured with plurality teeth. The second transmission gear 133c configured with plurality counter teeth and is configured to connect to the first transmission gear 133d through the interaction between those counter teeth and teeth of the first transmission gear 133d.

A printed circuit board (PCB) is arranged in the housing 131. The PCB comprises a switch 150, a signal unit and a processor. The switch 150 has a first switching position in which the motor 132 is configured to drive the first transmission gear 133d to rotate in a first rotation direction. The switch 150 has a second switching position in which the motor 132 is configured to drive the first transmission gear 133d to rotate in in a second rotation direction. The second rotation direction is opposite to the first rotation direction. For example, if the first rotation direction is a clockwise direction, the second rotation direction is a counter-clockwise direction.

The motor 132 has an inactive state in which the motor is not driving the first transmission gear 133d and an active state in which the motor drives the first transmission gear 133d.

Once the user uses the medicament delivery device 110, he/she will firstly connect and secure the proximal disposable part 20 to the distal reusable power pack 130, and switch on the main power of the delivery device, which for example may be done by attaching the proximal disposable part 20 or pressing a physical switch or a touch panel arranged on the housing 131. The user will then remove the protective cap (not show) and presses the delivery member cover 24 against the injection site. The distal end of the delivery member cover 24 acts as the trigger unit 24a, and switches the switch 150 by a flipping movement of the switch 150, pressing movement of the switch 150 or an electronic alignment connection in between (shown by FIG. 10), so that the switch is switched into the first switching position.

Once the switch 150 is in its first switching position, the motor 132 start to rotate and drive the first transmission gear 133d, so that the first transmission gear 133d is rotated in the first direction of rotation. The rotation of the first transmission gear 133d is transmitted to the second transmission gear 133c through the teeth connection between them. Due to the rotationally locked arrangement between the second transmission gear 133c, the central shaft 133b and the gear wheel 133a, the rotation of the second transmission gear 133c causes rotation of the gear wheel 133a.

The gear wheel 133a is configured to be rotated about an orthogonal axis extending along the central shaft 133b, and comprises a plurality of teeth, which are matched with counter teeth provided on the plunger body 34a so that, when the motor is driving the first transmission gear 133d in the first rotation direction, the rotation of the gear wheel 133a moves the plunger body 34a in the proximal direction. The force from the power unit 132 is thereby able to be transmitted to and move the plunger unit 34 in the proximal direction. The plunger head 34b moves the rubber stopper of the medicament container 40 in the proximal direction to expel a dose of medicament contained in the medicament container 40.

Once a predetermined dose of medicament has been fully delivered to the user, the motor 132 will be stop under the control of the processor and the processor produces a signal to the signal unit, so that the user becomes aware that the injection is completed.

The user will then remove the delivery device from the injection site, so that the delivery member cover 24 is moved in the proximal direction by the biasing member (not showed). Once the trigger unit 24a passes by the switch 150, it switches the switch 150 into the second switching position so that the motor 132 starts to drive the first transmission gear 133d in the second rotation direction. The rotation of the first transmission gear is transmitted to the second transmission gear 133c through their teeth connection, and again since the rotationally locked arrangement between the second transmission gear 133c, the central shaft 133b and the gear wheel 133a the rotation of the second transmission gear 133c causes the rotation of the gear wheel 133a.

The rotation of the gear wheel 133a causes the plunger body 34a and the plunger head 34b to be moved in the distal direction due to their teethed engagement. Once the plunger body 34a has been fully retracted to its initial position, the motor 132 is stopped again under the control of the processor, wherein the reset procedure is completed.

The plunger body 34a used in the first and the second embodiments can comprise a plurality of hingedly connected segments or a hard flexible wire with a plurality of teeth. The plunger body 34a and the plunger head 34b can be integral together as one component or two separate components hingedly connected to each other.

The embodiment as described above discloses components for easy manufacturing. However, it can also be modified under for simpler assembling. For example, the central shaft 133b and the gear wheel 133a can be integral as one component or the second transmission gear 133c, the central shaft 133b and the gear wheel 133a can be also integral as one component.

FIG. 11 illustrates a third embodiment of a medicament delivery device 210 for performing injections and having a proximal disposable part 20 and a distal reusable power pack 230. The proximal disposable part 20 used in the second embodiment is the same as the proximal disposable part 20 used in the first embodiment e.g. components, arrangements of components and functions are the same as described in the first embodiment, and can be connected to the distal reusable power pack 230 by the same connection as used in the first embodiment.

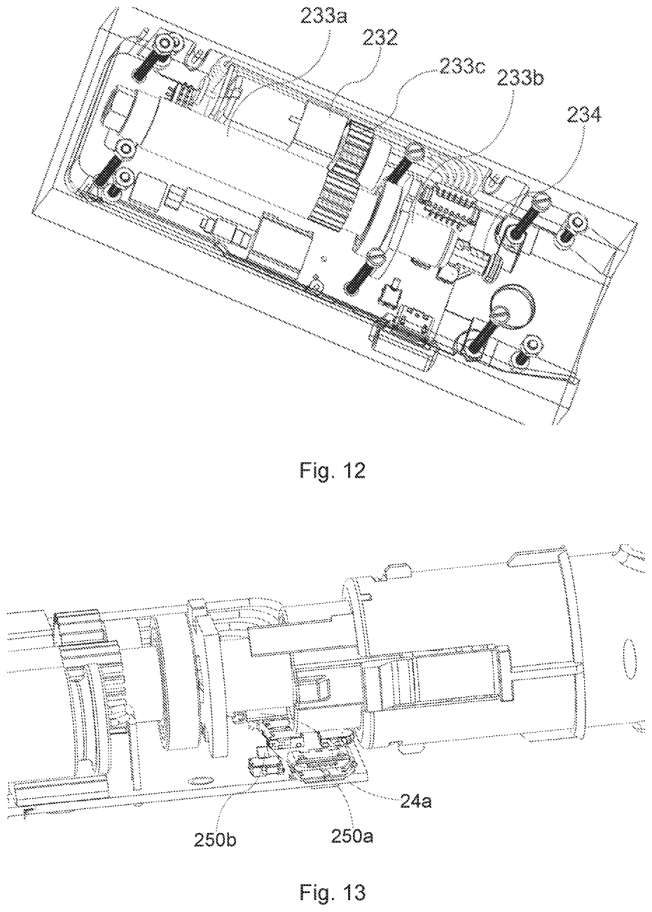

Turning to FIG. 12, the distal reusable power pack 230 comprises a housing 231, a power unit 232, a transmission gear 233c, a rotational drive unit 233, and a plunger unit 234. The power unit 232 is a motor connected to the transmission gear 233c.

The plunger unit 234 is configured to actuate the stopper of the medicament container 40 during the medicament ejection step. The rotational drive unit 233 is configured to connect the plunger unit 234 and moved the plunger unit 234 along a longitudinal direction. The power unit 232 is an element accumulated with the energy configured to transit the accumulated energy to the rotational drive unit 233.

The rotational drive unit 233 comprises a drive nut 233a and an insert 233b. The insert 233b is rotationally locked to the housing 231ä. The drive nut 233a has a set of teeth on its outer surface, which are arranged to mesh with a counter set of teeth of the transmission gear 233c.

The plunger unit 234 is an elongated plunger shaft with external threads and a slot arranged on its external surface. The insert 233b comprises inner threads, which are matched with the external threads of the plunger unit 234. The drive nut 233a comprises a ridge, which is matched with the external slot of the plunger unit 234. Thereby, the plunger unit 234 and the drive nut 233a are rotationally locked together but axially movable relatively.

The distal reusable power pack 230 comprises a PCB arranged in the housing 231. The PCB comprises a first switch 250a and a second switch 250b, a signal unit and a processor. The first switch 250a has a switching on position in which the motor 232 is configured to drive the transmission gear 233c in a first rotation direction. The second switch has a switch on position in which the motor 132 is configured to drive the transmission gear 233c in a second rotation direction. The second rotation direction is opposite to the first rotation direction. For example, if the first rotation direction is rotating clockwise the second rotation direction is rotating anti-clockwise.

The motor 232 has an inactive state in which the motor does not drive the transmission gear 233c, and an active state in which the motor is driving the transmission gear 233c.

Once the user is to use the medicament delivery device 210, he/she will firstly connect and secures the proximal disposable part 20 to the distal reusable power pack 230, and switch on the main power of the delivery device, which for example may be done by attaching the proximal disposable part 20 or pressing a physical switch or a touch panel arranged on the housing 231. The user will then remove the protective cap (not show); and presses the delivery member cover 24 against the injection site. The distal end of the delivery member cover 24 acts as a trigger unit 24a and switches the first switch 250a by a flipping movement of the first switch 250a, pressing movement of the first switch 250a or an electronic alignment connection in between; so that the first switch 250a is switched into the switching on position (shown by FIG. 13).

Once the first switch 250a is in its switching on position, the motor 232 starts to rotate and drive the transmission gear 233c in the first rotation direction, so that the transmission gear 233c is rotated about the longitudinal axis. The rotation of the transmission gear 233c is transmitted to the drive nut 233a through their teethed connection so that the drive nut 233a is rotated about an axis parallel with the said longitudinal axis.

The rotation of the drive nut 233a results in a rotation of the plunger unit 234 due to their rotationally locked engagement. Because of the thread engagement between the plunger unit 234 and the insert 233b, the plunger unit is moved by the rotational drive unit 233 in the proximal direction and moves the rubber stopper of the medicament container 40 in the proximal direction to expel a dose of medicament contained in the medicament container 40.

Once a predetermined dose of medicament has been fully delivered to the user, the motor 232 will be stop under the control of the processor and the processor generates a signal to the signal unit, so that the user becomes aware that the injection is completed.

The user will then remove the delivery device from the injection site, so that the delivery member cover 24 is moved in the proximal direction by the biasing member (not showed). Once the trigger unit 24a passes by the second switch 250b, it switches the second switch 250b into the switching on position so that the motor 232 starts to drive the transmission gear 233c in the second rotation direction.

The transmission gear 233c transmits the rotation to the drive nut 233a through their teethed connection, and again due to the rotationally locked arrangement between the drive nut 233a and the plunger unit 234 and the thread engagement between the plunger unit (234) and the insert (233b), the plunger unit (234) is moved in the distal direction. Once the plunger body 234 has been fully moved to its initial position, the motor 232 is stopped again under the control of the processor, and so the reset procedure is completed.

In an alternative embodiment, the drive nut 233a and the insert 233b are modified, such that the drive nut 233a with the inner thread surface to match with the external thread of the plunger unit 234 and rotationally arranged with the plunger unit 234. The insert 233b comprises the ridge to match with the slot on the external surface of the plunger unit 234 and is rotationally locked to the plunger unit 234 since the drive nut 233a is rotatable in relation to the plunger unit 234, but the rotation of the plunger unit 234 is limited by the ridge of the insert 233b; so that when the drive nut 233a is rotating, the plunger unit 234 is able to move in the proximal direction without rotation due to the thread engagement between the plunger unit 234 and the drive nut 233a and the ridge/slot engagement between the plunger unit 234 and the inset 233b.

The arrangements of the switch in the second and the third embodiments are interchangeable. The trigger of the injection and reset mechanism can be achieved by either one switch arrangement like described in the second embodiment or the two switches arrangement like described in the third embodiment.

The medicament container 40 can be a medicament container with an integral needle or a cartridge with a separate needle, connected to the medicament cartridge before injection. The medicament container 40 may for example only be sealed by the rubber stopper or with the rubber stopper and a plunger rod. The plunger head 34b or the plunger unit 234 disclosed in any of the embodiments can be arranged to bear against either the rubber stopper or the plunger rod.

The present concept has mainly been described above with reference to a few examples. However, as is readily appreciated by a person skilled in the art, other embodiments than the ones disclosed above are equally possible within the scope of the present concept, as defined by the appended claims.

1-15. (canceled)

16: A medicament delivery device comprising:
a proximal disposable part arranged with a body and a delivery member cover telescopically arranged in relation to the body; wherein the proximal disposable part is configured to receive a medicament container;
a distal reusable power pack detachably arranged with the proximal disposable part;
wherein said delivery member cover has an extension position in which the delivery member cover is extended in the proximal direction to cover a medicament delivery member of the medicament container, and a retraction position in which the delivery member cover is retracted in the distal direction to expose said medicament delivery member;
wherein said distal reusable power pack comprises a power unit, a rotational drive unit and a plunger unit;
wherein said rotational drive unit is powered by said power unit and is configured to move said plunger unit upon being triggered;
wherein said plunger unit is configured to move a stopper of the medicament container in the proximal direction upon being moved by said rotational drive unit, so that a medicament contained in the medicament container is expelled;
wherein the delivery member cover comprises a trigger configured to trigger said rotational drive unit to move said plunger unit when said delivery member cover is moved distally to the retraction position.

17: The medicament delivery device according to claim 16, wherein said power unit is a spring, the spring being configured to deliver a spring force along its circumferential direction.

18: The medicament delivery device according to claim 16, wherein said power unit is a motor.

19: The medicament delivery device according to claim 16, wherein said distal reusable power pack comprises a housing, said housing being configured to receive said rotational drive unit and said power unit, wherein said housing further comprises a locking element configured to prevent said rotational drive unit from rotating in relation to said housing in a locking position.

20: The medicament delivery device according to claim 19, wherein said trigger is configured to release said locking element to move out from the locking position, thereby triggering said rotational drive unit to move the plunger unit.

21: The medicament delivery device according to claim 18, wherein the distal reusable power pack further comprises a switch; wherein said trigger is configured to trigger said motor by switching the switch.

22: The medicament delivery device according to claim 17, wherein said distal reusable power pack further comprises a reset knob arranged to rotate the rotational drive unit against the restoring force of the spring.

23: The medicament delivery device according to claim 22, wherein the rotation of said reset knob actuates said plunger unit to move in the distal direction in relation to said proximal disposable part by the connection between the plunger unit and the rotational drive unit.

24: The medicament delivery device according to claim 18, wherein said motor is configured to reciprocate the movement of said plunger unit.

25: The medicament delivery device according to claim 16, wherein said rotational drive unit comprises a rotational gear wheel configured to rotate about an orthogonal axis of the distal reusable power pack.

26: The medicament delivery device according to claim 16, wherein said rotational drive unit comprises a drive nut configured to rotate about a longitudinal axis of the distal reusable power pack.

27: The medicament delivery device according to claim 25, wherein said plunger unit has a plurality of teeth, said plunger unit being arranged to connect with said gear wheel by means of said teeth.

28: The medicament delivery device according to claim 25, wherein said distal reusable power pack further comprises a damper gear arranged to connect to said gear wheel, said damper gear being configured to slow down the rotational speed of said gear wheel.

29: The medicament delivery device according to claim 26, wherein said plunger unit comprises a thread on its external surface.

30: The medicament delivery device according to claim 16, wherein said delivery member cover comprises a rib and said proximal disposable part comprises a rotator configured with a locking tongue, said rotator being configured to be rotated by a linear movement of the delivery member cover, wherein said rotation aligns the locking tongue with said rib, so that the delivery member cover is prevented from moving to the retraction position.

31: A medicament delivery device comprising:
a proximal disposable part comprising a body and a delivery member cover axially having a longitudinal axis and movable relative to the body, wherein the proximal disposable part comprises a medicament container; and
a distal reusable power pack detachably arranged with the proximal disposable part,
wherein the delivery member cover has an extension position in which the delivery member cover is extended in the proximal direction to cover a medicament delivery member fixed to the medicament container, and a retraction position in which the delivery member cover is moved distally relative to the housing to expose the medicament delivery member,
wherein the distal reusable power pack comprises a power unit, a rotational drive unit, and a plunger unit that moves transverse to the longitudinal axis to move a stopper within the medicament container in the proximal direction when moved by the rotational drive unit, and
wherein movement of the delivery member cover to the retraction position causes the rotational drive unit to move the plunger unit.

32: The medicament delivery device of claim 31 further, wherein the delivery member cover comprises a rib and the proximal disposable part comprises a rotator, where movement of the delivery member cover to the retraction position causes the rib to engage the rotator to rotate the rotator and to activate the power unit such that rotational drive unit rotates.

33: The medicament delivery device of claim 32, wherein the rotator comprises a locking tongue that is rotated by a linear movement of the delivery member cover such that the locking tongue align with the rib when the delivery member cover has moved from the retraction position to the extension position whereby the delivery member cover is prevented from moving back to the retraction position.

34: The medicament delivery device of claim 31, wherein the plunger unit has a plurality of teeth that engage with a gear wheel fixed to the rotational drive unit such that the gear wheel rotates along an axis transverse to the longitudinal axis.

35: The medicament delivery device of claim 31 further comprising a locking element that when in a locking position prevents the rotational drive unit from rotating relative to the housing, wherein a trigger operatively associated with the delivery member cover releases the locking element from the locking position to cause the rotational drive unit to move the plunger unit.
